(19) United States
(12) Patent Application Publication
(10) Pub. No.: US 2026/0252231 A1
(43) Pub. Date: Aug. 27, 2026

(54) PLANNING AND/OR CONTROL SYSTEM FOR A NEUROMODULATION SYSTEM

(71) Applicant: ONWARD MEDICAL N.V., Eindhoven (NL)

(72) Inventors: Robin BROUNS, Eindhoven (NL); Jurriaan BAKKER, Eindhoven (NL); Miroslav CABAN, Renens (CH)

(73) Assignee: ONWARD MEDICAL N.V., Eindhoven (NL)

(21) Appl. No.: 19/644,675

(22) Filed: Apr. 10, 2026

Related U.S. Application Data
(63) Continuation of application No. 16/953,322, filed on Nov. 19, 2020, now Pat. No. 12,613,624.

(30) Foreign Application Priority Data
Nov. 19, 2019 (EP) 19209911.7

(51) Int. Cl. G06F 3/04847; A61N 1/05

(52) U.S. Cl. CPC G06F 3/04847; A61N 1/0551; A61N 1/36062; A61N 1/36125; A61N 1/36128; A61N 1/36185; A61N 1/36189; A61N 1/37247; A61N 1/37282; G06F 3/0482; G16H 20/30; G16H 40/63; G06F 3/0488

(57) ABSTRACT

A planning and/or control system for providing stimulation is disclosed. The system can include a graphical presentation module configured and arranged for providing graphical information about an electrode array comprising multiple electrodes and/or an implantation side for the electrode array comprising at least one target area, a selection module configured and arranged for determining a stimulation zone and/or a stimulation direction on the electrode array comprising at least one electrode and/or for individually selecting at least one electrode and/or for selecting at least one target area, and a calculation module configured and arranged for determining a contribution of currents provided by electrodes of the stimulation zone and/or stimulation direction on the electrode array and/or the at least one electrode selected and/or to the at least one target area selected.

### CROSS REFERENCE TO RELATED APPLICATION

The present application claims priority to European Patent Application No. EP 19209911.7 filed on Nov. 19, 2019. The entire contents of the above-listed application is hereby incorporated by reference for all purposes.

### TECHNICAL FIELD

Disclosed embodiments relate to control systems for tissue stimulating systems, in some embodiments a planning and/or control system for a system for providing neuromodulation, especially neurostimulation for a patient.

### BACKGROUND AND SUMMARY

The disclosed embodiments further relate to a method for planning neuromodulation, and in some embodiments neurostimulation for a patient.

The spinal cord is an integral part of the central nervous system (CNS). Spinal cord injury (SCI), but also other disorders (e.g. stroke, multiple sclerosis, autonomic failure, autonomic neuropathy or cancer of the neurological tissue which impair operation of descending sympathetic pathways that normally facilitate control of autonomic functions) result in motor deficits. For instance, SCI interrupts the communication between the spinal cord and supraspinal centers, depriving these sensorimotor circuits from the excitatory and modulatory drives necessary to produce movement. However, SCI also results in sensory deficits and in autonomic dysfunctions. In particular, SCI results in disconnection of some, most, or all descending sympathetic pathways that carry signals responsible for regulating arterial blood pressure, heart rate and/or gut and/or bladder function.

Spinal cord stimulation (SCS) is a well-established neuromodulatory therapy not only for restoring locomotion/motoric function after spinal cord injury or central nervous diseases, but also for treating inter alia pain and/or restoring autonomic function.

Neuromodulation, in particular neurostimulation, in particular SCS can be applied to a subject by a neuromodulation system comprising at least one electrode array comprising at least one electrode. Neuromodulation systems may further comprise at least one of a controller, e.g. a microcontroller, a processor, e.g. a microprocessor, a pulse generator, in some embodiments an implantable pulse generator, a sensor, a communication module, a telemetry module.

The electrode array, e.g. comprised in a lead paddle, can be applied for percutaneous electrical stimulation, transcutaneous electrical nerve stimulation (TENS), epidural electrical stimulation (EES), subdural electrical stimulation (SES), functional electrical stimulation (FES) and/or all neurostimulation and/or muscle stimulation applications that use at least one electrode array and/or at least one electrode.

Lead paddles are for example described in U.S. Pat. No. 8,108,051B2, US 2013/0096662 A1, US 2012/0006793A1 and EP3013411A1.

A neuromodulation system, especially a neurostimulation system for a patient suffering from motoric dysfunction and/or autonomic dysfunction may require programming to define which stimulation settings can be used to evoke certain muscles or muscle groups. Such muscles and/or muscle groups may be responsible for locomotion of the arms or legs, and/or responsible for e.g. bowel movement, sphincter control, bladder control and/or sexual function. Such programming of stimulation parameters may be performed by a user, such as a clinical professional, a physiotherapist and/or the patient himself, and facilitated by a computer-driven application. Neurostimulation, in particular multi-channel and/or variable neurostimulation may require an interface to create the stimulation program and a stimulation system to deliver the stimulation. A user may select specific electrodes via a user interface on which he would like to perform stimulation for a patient who's implanted with the lead comprising the electrode array.

EP3527258A1 discloses a system for an electrical neurostimulator coupled to a plurality of electrodes, comprising: a user-controlled input device configured for generating directional control signals; and control circuitry configured for sequentially defining a plurality of different ideal bipole/tripole configurations relative to the plurality of electrodes in response to the directional control signals, generating a plurality of stimulation parameter sets respectively corresponding to the plurality of ideal bipole/tripole configurations, each stimulation parameter set defining relative amplitude values for the plurality of electrodes that emulate the respective ideal bipole/tripole configuration, and instructing the neurostimulator to convey electrical energy to the plurality of electrodes in accordance with the plurality of stimulation parameter sets.

EP3285855B1 discloses a system for delivering neurostimulation including a programming control circuit and a user interface. The programming control circuit may be configured to generate stimulation parameters controlling delivery of neurostimulation pulses according to one or more stimulation waveforms associated with areas of stimulation each defined by a set of electrodes. The neurostimulation pulses are each delivered to an area of stimulation. The user interface may include a display screen and an interface control circuit. The interface control circuit may be configured to define the one or more stimulation waveforms and the areas of stimulation and may include a stimulation frequency module configured to display a stimulation rate table on the display screen. The stimulation rate table may present stimulation frequencies associated with each of the areas of stimulation for selection by a user.

Currently, leads comprising an electrode array comprising multiple electrodes with an intended use for neuromodulation offer a limited range of freedom since their resolution is confined by the number of electrodes. Options such as unipolar and multipolar stimulation increase field-steering capabilities, but the user is still limited by the fixed set of electrodes that he or she can pick from.

Therefore, the present disclosure provides a neuromodulation system which allows increasing neuromodulation options on a lead comprising an electrode array comprising electrodes.

This increasing of the neuromodulation options may be solved according to the disclosed embodiments by a planning and/or control system for a system for providing neuromodulation, including neurostimulation, at least comprising:
a graphical presentation module configured and arranged for providing graphical information about an electrode array comprising multiple electrodes and/or an implantation side for the electrode array comprising at least one target area,
a selection module configured and arranged for determining a stimulation zone and/or a stimulation direction on the electrode array comprising at least one electrode and/or for individually selecting at least one electrode and/or for selecting at least one target area,
a calculation module configured and arranged for determining a contribution of currents provided by electrodes of the stimulation zone and/or stimulation direction on the electrode array and/or the at least one electrode selected and/or to the at least one target area selected.

Consistent with the disclosed embodiments, the amount of stimulation options and/or stimulation resolution of a lead comprising an electrode array may be increased by using electrode and/or current weighting. Thus, the options for choosing stimulation options may no longer be constricted by selecting discrete electrodes (that means separate electrodes or one or more single electrode(s)) on a lead and/or electrode array but leverages the wide freedom of current weighting/balancing and presenting this in an intuitive way to a user using the system. Alternatively, and/or additionally, combining stimulation options with imaging may allow a user to create a root for stimulation or steering current to more naturally stimulate nerves rather than creating homogenous fields. The use of a graphical presentation module, a selection module and a calculation module combined into one strategy and made available for a system for providing neuromodulation, including neurostimulation may reduce limitations of stimulation options with current leads and/or electrode arrays and provide the patient the best possible stimulation.

In some embodiments, the system may use anatomical information about the dorsal root trajectory, to adapt the weighing of electrode currents, to steer the field, such (parallel to the trajectories) that maximum activation is obtained at target roots, while avoiding activation of the non-targeted roots.

The graphical presentation module may provide graphical information about an electrode array comprising physical electrodes and/or virtual electrodes. In some embodiments, physical electrodes may reflect physical electrodes of a corresponding neuromodulation system related to the system for planning and/or control system. In some embodiments, virtual electrodes may be represented by the graphical presentation module of the system only and may not reflect physical electrodes of a corresponding neuromodulation system related to the panning and/or control system according to the disclosed embodiments. In other words, virtual electrodes may be introduced by the system without physically introducing more physical electrodes. In some embodiments, any number of virtual electrodes may be introduced in addition to the physical electrodes. In some embodiments, virtual electrodes presented to a user may be more understandable and graspable than current-weighting or current-steering in percentages or absolute numbers. Overall, this enhances the options for choosing stimulation options.

In some embodiments, each virtual and/or physical electrode may be controlled independently. In some embodiments, this may enable that each virtual and/or physical electrode may be characterized by a different frequency and/or waveform.

In some embodiments, the user may be e.g. a therapist, a physiotherapist, a physician, a trainer, a medical professional, a patient and/or any person related to the patient.

In some embodiments, the target area may be or may comprise fictious and/or realistic anatomical conditions of a mammal, in some embodiments a human being, such as the patient himself or a healthy individual. In some embodiments, the target area may be or may comprise at least one target nerve and/or nerve fiber and/or dorsal root and/or area of the spinal cord and/or tissue and/or area related to the spinal cord and/or muscle fiber and/or muscle.

In some embodiments, the target area could be augmented with an approximate neuronal model, e.g. neuronal fibers passing through dorsal roots. In some embodiments, the system may calculate contribution of currents based on neuronal simulations and/or anatomical conditions.

The selection module may be or may comprise a user interface. In some embodiments, the user interface may be a graphical user interface. In some embodiments, the selection module may be an input module. The selection module may comprise a mouse and/or a trackball and/or a joystick, a display and/or a touch screen and/or a touch pad and/or an acoustic signal and/or acoustic tone and/or a verbal command input. In some embodiments, the selection module may be configured and arranged for allowing a user to actuate at least one control element, including but not limited to axes, points, knots, buttons, arrows, hand signals, emojis, crosses and/or windows and/or text and/or shortcuts in order to modify graphical information.

In some embodiments, the selection module may enable to select information and/or links and/or strategies provided by the graphical presentation module.

In some embodiments, the stimulation zone may be or may comprise an area on a lead and/or electrode array comprising multiple electrodes, wherein the area comprises at least one electrode.

In some embodiments, a stimulation zone may also be or may comprise a stimulation vector and/or a stimulation direction and/or at least one single electrode.

The calculation module may be or may comprise at least one algorithm. In some embodiments, the calculation module may translate a selected stimulation zone and/or stimulation direction and/or the one or more electrodes individually selected and/or the at least one target area selected into a certain weighting, such as current weighting, on a real electrode array comprising electrodes, for stimulating a patient.

The calculation module may be configured and arranged for determining an equal contribution of currents provided by the electrodes of the stimulation zone and/or stimulation direction and/or the one or more electrodes individually selected and/or to the at least one target area selected. In some embodiments, a user may select a stimulation zone and/or a stimulation direction and/or one or more electrodes individually and current sourcing may be distributed equally over all electrodes and/or anodes selected. Alternatively, and/or additionally, a user may select at least one target area and current sourcing may be distributed equally over the electrodes located at the at least one target area. In some embodiments, this has the advantage that differences in excitation and/or excitability of different target nerves during equal stimulation may identified, which finally may enable optimizing stimulation protocols.

The calculation module may be configured and arranged for determining a weighted contribution of currents provided by the electrodes of the stimulation zone and/or stimulation direction and/or the at least one electrode individually selected and/or to the at least one target area selected. Overall, weighted contribution of currents, compared to homogenous fields, may enable more effective and closer to natural stimulation. In some embodiments, weighted contribution may direct a current to overcome neurological activity thresholds in an optimized matter. In some embodiments, the second spatial derivative, tangential to a neuron/nerve may be the driving force behind activation. A simple field normal to a fiber may not lead to predictive activation, yet a tangent directional field may lead to a predictive activation.

In some embodiments, for a stimulation direction, such as a stimulation vector, the weighting of electrodes may comprise distance and/or direction, in some embodiments with the origin of the stimulation direction as the reference.

The calculation module may be configured and arranged for determining the weighted contribution of currents provided by the electrodes of the stimulation zone and/or stimulation direction by calculating the Euclidean distance from an electrode to the stimulation zone and/or stimulation direction and/or to at least one point of the stimulation zone and/or stimulation direction.

In some embodiments, the Euclidean distance may be the ordinary distance between two points, here two electrodes and/or points of two electrodes. In some embodiments, it may be the straight-line distance between two points and/or two electrodes and/or two points of electrodes. In some embodiments, in a stimulation zone comprising more than two electrodes, this may have the advantage that the distance and the weighting of two electrodes is not affected by the addition of a third electrode to the analysis. Overall, weighting of each electrode of a stimulation zone and/or stimulation direction, based on the absolute and/or relative distance of the respective electrode of the stimulation zone and/or stimulation direction to one point of the stimulation zone and/or stimulation direction may be enabled. This may provide decreased stimulation intensity, the further an electrode of the stimulation zone is positioned away from a specific stimulation center of a stimulation zone. This may enable close to natural stimulation.

In some embodiments, the Euclidean distance may be the ordinary distance between two points, here e.g. two nerves and/or nerve fibers and/or parts of nerves and/or parts of nerve fibers of at least one target area. In some embodiments, it is the straight-line distance between e.g. two nerves and/or nerve fibers and/or parts of nerves and/or parts of nerve fibers of at least one target area. Overall, weighting of nerves and/or nerve fibers and/or parts of nerves and/or parts of nerve fibers of at least one target area, based on the absolute and/or relative distance of the respective nerve and/or nerve fiber and/or part of a nerve and/or part of a nerve fiber of the target area to one point of the target area may be enabled. This may enable that e.g. decreased stimulation intensity is provided, the further a nerve and/or nerve fiber and/or part of nerve and/or part of nerve fiber of at least one target area is from a specific stimulation center of a target area. This may enable close to natural stimulation.

The calculation module may be configured and arranged for determining the weighted contribution of currents provided by the electrodes of the stimulation zone and/or the stimulation direction and/or at least one electrode individually selected based on a generated field of neighbor electrodes. In some embodiments, this may enable soft transitions of the current strengths of adjacent electrodes. Overall, this may enable close to natural stimulation.

The calculation module may be configured and arranged for determining the weighted contribution of currents provided by the electrodes of the stimulation zone and/or the stimulation direction and/or the at least one electrode individually selected and/or to the at least one target area selected by a numerical method. In some embodiments, this approach may use a personalized patient model comprising the targeted and unwanted nerve locations (crosstalk), and which has computed for N electrode configurations the 3D potential distributions, as well as the activation potentials and/or selectivity indices. In some embodiments, based on the stimulation zone and/or the stimulation direction and/or the at least one electrode individually selected and/or the at least one target area selected the numerical method may optimize the weighting of electrode intensities for maximizing a selectivity index, or for maximizing the target while minimizing sensitive areas. Overall, this may enable stimulation adapted to patient specific needs, i.e. patient specific stimulation.

In some embodiments, different frequencies and/or pulse widths may be applied to different electrodes to create a stimulation zone. In some embodiments, a user may select a certain zone and intends the zone to have 130 Hz stimulation, which may then be achieved by delivering differing stimulation frequencies to its surrounding electrodes, and the frequency of 130 Hz in the stimulation zone may be achieved by amplification (overlapping, or constructive interference) or cancellation interference.

Further, the system may determine for which intended muscles/roots, a certain waveform should be used, e.g. a low frequency, a higher frequency, or a burst (e.g. triplets/quadruplets, with different burst frequency) waveform. In this case, the system may use knowledge of neurophysiology, i.e. which muscles (agonists/antagonists) and/or roots to determine if a higher frequency, or a burst frequency is required to increase activation of target muscles and/or roots, while keeping unwanted muscles and/or roots less exposed.

The calculation module may be configured and arranged to feature an algorithm to determine the weighted contribution of currents to the benefit of power efficiency. This algorithm may be appended to any of the previously mentioned calculation module implementations. This algorithm may interpret the input weighted electrode configuration and optimize it such that a new configuration is created that creates a considerably similar electrical field with the same effect on the effectuated neural tissue, yet with better power efficiency. In some embodiments, with the increased stimulation freedom offered to the system by the ‘Virtual Electrodes’ concept, the search-space for more power-efficient settings to the algorithm is also increased. In addition to the algorithm altering the current weighting, the algorithm may further achieve power-efficiency effects by combining this with reduction or increase of the amount of allocated electrodes (to reduce impedance), or may update the required stimulation current or total charge required to achieve the neurostimulation.

The system may further comprise at least one computer-assisted module configured and arranged for at least partially automatically determining a stimulation zone and/or a stimulation direction on the electrode array comprising at least one electrode and/or for at least partially automatically selecting at least one electrode, optionally based on medical imaging superimposing. In some embodiments, medical images may be obtained by MRI, CT, X-Ray, photography, etc. Alternatively, and/or additionally, anatomical models could be used. In some embodiments, this may enable finding optimal zones and/or targets for stimulation, specifically adapted to the patient's anatomy and needs.

In other words, the stimulation direction and/or stimulation zone on an electrode array comprising at least one electrode may be either selected manually by a user or automatically or semi-automatically by identifying a target area from anatomical data.

Accordingly, embodiments of the present disclosure provide: On the visualization of the electrode overlapped on the anatomical model (with roots visible), the desired stimulation zone may be drawn (and direction) in order to target the roots of interest.

Furthermore, this zone may be determined automatically when a software identifies the roots automatically from the anatomical model (e.g. MRI).

The system may further comprise at least one of a display, a controller, a programmer, a communication module, a telemetry module, a stimulation device, an electrode, a sensor and/or a sensor network.

In some embodiments, the system may be a closed-loop system or an open-loop system.

In some embodiments, the system allows both closed-loop or open loop functionality. In this regard, the user may switch between these options or there may be routines or control elements that can do or propose such a switch from closed-loop to open-loop and vice versa.

In some embodiments, the system may be related to a system for providing neuromodulation, in some embodiments neurostimulation to a patient. The system for providing neuromodulation, in some embodiments neurostimulation to a patient may comprise at least one of a controller, a programmer, a communication module, a telemetry module, a stimulation device comprising an electrode array comprising multiple electrodes, a sensor and/or a sensor network.

According to the disclosed embodiments a method is disclosed, the method being performed with the system described above.

In some embodiments, a method for planning neuromodulation, especially neurostimulation, may comprise at least the steps of:
providing graphical information about an electrode array comprising multiple electrodes and/or an implantation side for the electrode array comprising at least one target area,
determining a stimulation zone and/or a stimulation direction on the electrode array comprising at least one electrode and/or individually selecting at least one electrode and/or selecting at least one target area,
determining a contribution of currents provided by electrodes of the stimulation zone on the electrode array and/or the at least one electrode selected and/or to the at least one selected target area.

In some embodiments, the graphical information about the electrode array may comprise actual physical electrodes and/or virtual electrodes.

The method may further comprise the step of determining an equal contribution of currents provided by the electrodes of the stimulation zone and/or the stimulation direction and/or at least one electrode individually selected and/or to the at least one target area selected.

In some embodiments, the method may further comprise the step of determining a weighted contribution of currents provided by the electrodes of the stimulation zone and/or the stimulation direction and/or at least one electrode individually selected and/or to the at least one target area selected.

The method may further comprise the step of determining the weighted contribution of currents provided by the electrodes of the stimulation zone and/or the stimulation direction by calculating the Euclidean distance from an electrode to the stimulation zone and/or the stimulation direction and/or to at least one point of the stimulation zone and/or stimulation direction.

The method may further comprise the step of determining the weighted contribution of currents provided by the electrodes of the stimulation zone and/or the stimulation direction and/or the at least one electrode individually selected based on a generated field of neighbor electrodes.

The method may further comprise the step of determining the weighted contribution of currents provided by the electrodes of the stimulation zone and/or the stimulation direction and/or at least one electrode individually selected and/or to the at least one target area selected by a numerical method.

The method may further comprise the step of least partially automatically determining a stimulation zone and/or a stimulation direction on the electrode array comprising at least one electrode and/or for at least partially automatically selecting at least one electrode, optionally based on medical imaging superimposing.

The method may further comprise the step of featuring an algorithm to determine the weighted contribution of currents to the benefit of power efficiency.

It is to be understood that both the foregoing general description and the following detailed description are exemplary and explanatory only and are not restrictive of the disclosed embodiments or the scope of the inventions as claimed. The concepts in this application may be employed in other embodiments without departing from the scope of the inventions.

### BRIEF DESCRIPTION OF THE FIGURES

Further details and advantages of the disclosed embodiments shall now be disclosed in connection with the drawings. The accompanying drawings, which are incorporated in and constitute a part of this specification, illustrate several embodiments consistent with the disclosure and together with the description, serve to explain the principles of the disclosure. In the drawings:

FIG. 1 shows a schematic overview of an embodiment of the planning and/or control system for a system for providing neuromodulation, especially neurostimulation according to the disclosed embodiments, with which the method according to the disclosed embodiments may be performed;

FIG. 2 shows an example of an equal contribution of currents provided by individually selected electrodes of an electrode array, according to the disclosed embodiments;

FIG. 3 shows an example of weighted contribution of currents provided by electrodes of a determined stimulation zone of an electrode array, according to the disclosed embodiments;

FIG. 4 shows an example of an embodiment of an electrode array, comprising virtual electrodes, according to the disclosed embodiments;

FIG. 5 shows an example of determining a stimulation direction, according to the disclosed embodiments;

FIG. 6 shows two examples of graphical information provided by the graphical presentation module, combining an electrode array with a target area, according to the disclosed embodiments;

FIG. 7 shows a further example of graphical information provided by the graphical presentation module, combining an electrode array with at least one possible target area, according to the disclosed embodiments; and

FIG. 8 shows a further example of graphical information provided by the graphical presentation module, according to the disclosed embodiments.

### DETAILED DESCRIPTION

Reference will now be made in detail to exemplary embodiments, discussed with regards to the accompanying drawings. In some instances, the same reference numbers will be used throughout the drawings and the following description to refer to the same or like parts. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

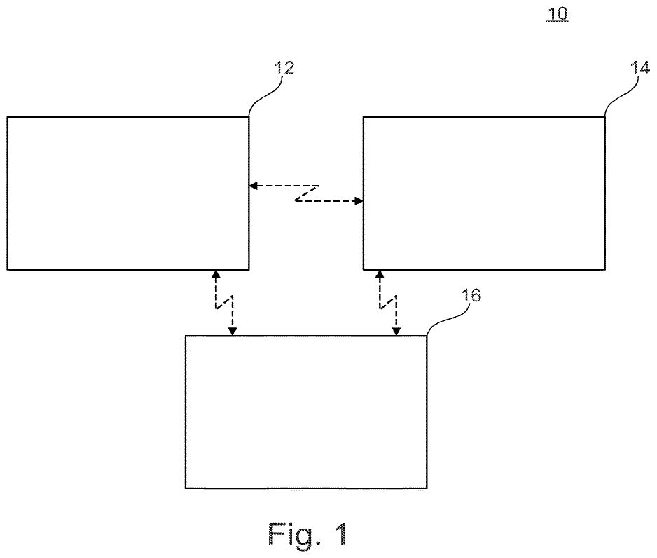

FIG. 1 shows a schematic overview of an embodiment of the planning and/or control system 10 for a system for providing neuromodulation, especially neurostimulation according to the disclosed embodiments, with which the method according to the disclosed embodiments may be performed.

The system 10 comprises a graphical presentation module 12, a selection module 14, and a calculation module 16. In some examples, the graphical presentation module 12, the selection module 14, and the calculation module 16 may be present on a single device, or one or more of the graphical presentation module 12, the selection module 14, and the calculation module 16 may be present on separate devices. The device(s) comprising the graphical presentation module 12, the selection module 14, and the calculation module 16 may include memory, one or more processors, and a communication subsystem, though other components and modules may also be included as known to those of skill in the art. In some aspects, the device(s) comprising the graphical presentation module 12, the selection module 14, and the calculation module 16 may be coupled to a user input device, a display, an electrode array comprising one or more electrodes, and/or other peripheral components.

Collectively, the various tangible components or a subset of the tangible components of the planning and/or control system 10 may be referred to herein as “logic” configured or adapted in a particular way, for example as logic configured or adapted with particular software, hardware, or firmware and adapted to execute computer readable instructions. The processors may be single core or multicore, and the programs executed thereon may be configured for parallel or distributed processing. The processors may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration, that is, one or more aspects may utilize ubiquitous, convenient, on-demand network access to a shared pool of configurable computing resources that can be rapidly provisioned and released with minimal management effort or service provider interaction. Clouds can be private, public, or a hybrid of private and public, and may include Infrastructure as a Service (IaaS), Platform as a Service (PaaS) and Software as a Service (SaaS). In some aspects, logic and memory may be integrated into one or more common devices, such as an application specific integrated circuit, field programmable gate array, or a system on a chip.

In some embodiments, one or more of the device(s) of the planning and/or control system 10 may be any computing or mobile device, for example, mobile devices, tablets, laptops, desktops, PDAs, and the like, as well as virtual reality devices or augmented reality devices. Thus, in some embodiments, the device(s) may include a display and thus a separate display or user input device may not be necessary. In other aspects, the device(s) may be coupled to a plurality of displays.

The memory generally comprises a random-access memory (“RAM”) and permanent non-transitory mass storage device, such as a hard disk drive or solid-state drive. The memory may store an operating system as well as the various modules and components discussed herein. It may further include devices which are one or more of volatile, non-volatile, dynamic, static, read/write, read-only, random access, sequential access, location addressable, file addressable and content addressable.

The communication subsystem may be configured to communicatively couple the modules within a device as well as communicatively coupling a device with one or more other computing and/or peripheral devices. Such connections may include wired and/or wireless communication devices compatible with one or more different communication protocols including, but not limited to, the Internet, a personal area network, a local area network (LAN), a wide area network (WAN) or a wireless local area network (WLAN). For example, wireless connections may be WiFi, Bluetooth®, IEEE 802.11, and the like.

In some embodiments, the graphical presentation module 12 is configured and arranged for providing graphical information about an electrode array A comprising multiple electrodes E, V and/or an implantation side for the electrode array A comprising at least one target area X.

The system 10 further comprises the selection module 14. In some embodiments, the selection module 14 is configured and arranged for determining a stimulation zone Z and/or a stimulation direction D on the electrode array A comprising at least one electrode E, V and/or for individually selecting at least one electrode E, V and/or for selecting at least one target area X.

The system 10 further the calculation module 16. In some embodiments, the calculation module 16 is configured and arranged for determining a contribution of currents provided by electrodes E, V of the stimulation zone Z and/or stimulation direction D on the electrode array A and/or the at least one electrode E, V selected and/or a to the at least one target area X selected.

In some embodiments, the graphical presentation module 12, the selection module 14 and the calculation module 16 may be connected. In some embodiments, the graphical presentation module 12, the selection module 14 and the calculation module 16 may be connected via a bidirectional connection. In some embodiments, the graphical presentation module 12, the selection module 14 and the calculation module 16 may be connected via a wireless link. In various embodiments, a unidirectional and/or cable-bound connection between the graphical presentation module 12, the selection module 14 and/or the calculation module 16 may be used.

In some embodiments, the graphical presentation module 12 provides graphical information about an electrode array A comprising multiple electrodes E, V. In various embodiments, the graphical presentation module 12 may provide additionally and/or alternatively graphical information abound an implantation side for the electrode array A comprising at least one target area X. In some embodiments, the graphical information about the implantation site could be patient-specific data. In some embodiments, the graphical information about the implantation site could be patient-specific MRI data, X-Ray data, pictures obtained during surgery, etc.

In some embodiments, the selection module 14 can be configured to determines a stimulation zone Z. In some embodiments, the selection module 14 can be configured to determine a stimulation zone Z based on a user input. In some embodiments, the selection module 14 may comprise a user interface. In various embodiments, the selection module 14 could additionally and/or alternatively determine a stimulation direction D on the electrode array A comprising at least one electrode E, V and/or for selecting at least one target area X, based on user input.

In some embodiments (not depicted in FIG. 1), system 10 could further comprise at least one of a display, a controller, a programmer, a communication module, a telemetry module, a stimulation device, an electrode, a sensor and/or a sensor network, as described above. For example, each of the modules described herein could be included as part of or coupled to a controller, where the controller includes a non-transitory memory (e.g., the memory described above) storing instructions that are executable by a processor (e.g., the processor described above) to perform the functions described herein.

In some embodiments (not depicted in FIG. 1), graphical presentation module 12 could in general provide graphical information about an electrode array A comprising actual physical electrodes E and/or virtual electrodes V. Graphical presentation module 12 could be configured to enable selection of one or more virtual electrodes V for use in stimulation. In some embodiments, selection of a virtual electrode may be mathematically equivalent to selection of physical electrodes and the assignment of current steering parameters (e.g., current weights, steering percentages, or similar percentages or absolute numbers describing the distribution of current) to the selected physical electrodes. However, configuring stimulation through the selection of one or virtual electrodes V may be conceptually simpler and easier to understand.

In some embodiments (not depicted in FIG. 1), calculation module 16 could be configured and arranged to determine an equal contribution of currents provided by the electrodes E, V of the stimulation zone Z and/or the stimulation direction D and/or the one or more electrodes E, V individually selected and/or to the at least one selected target area X.

In some embodiments (not depicted in FIG. 1), calculation module 16 could be configured and arranged to determine a weighted contribution of currents provided by the electrodes E, V of the stimulation zone Z and/or stimulation direction D and/or the at least one electrode E, V individually selected and/or to the at least one selected target area X.

In some embodiments (not depicted in FIG. 1), calculation module 16 could be configured and arranged to determine the weighted contribution of currents provided by the electrodes E, V of the stimulation zone Z and/or stimulation direction D by calculating the Euclidean distance from an electrode E, V to the stimulation zone Z and/or stimulation direction D and/or to at least one point of the stimulation zone Z and/or stimulation direction D.

In some embodiments (not depicted in FIG. 1), calculation module 16 could be configured and arranged to determine the weighted contribution of currents provided by the electrodes E, V of the stimulation zone Z and/or the stimulation direction D and/or at least one electrode E, V individually selected based on a generated field of neighbor electrodes E, V.

In some embodiments (not depicted in FIG. 1), calculation module 16 could be configured and arranged to determine which neighboring electrodes E, V, given what weighted contribution of currents, can create a specified stimulation vector and/or a stimulation direction D. In some embodiments (not depicted in FIG. 1), calculation module 16 could be configured and arranged for determining the weighted contribution of currents provided by the electrodes E, V of the stimulation zone Z and/or the stimulation direction D and/or the at least one electrode E, V individually selected and/or to the at least one target area X selected by a numerical method. In some embodiments, the a numerical method could use a personalized patient model M, e.g. a MRI based patient model M, that includes both targeted and unwanted (cross-talk) nerve locations, and which has computed for N electrode E, V configurations the 3D potential distributions, as well as the activation potentials/selectivity indices.

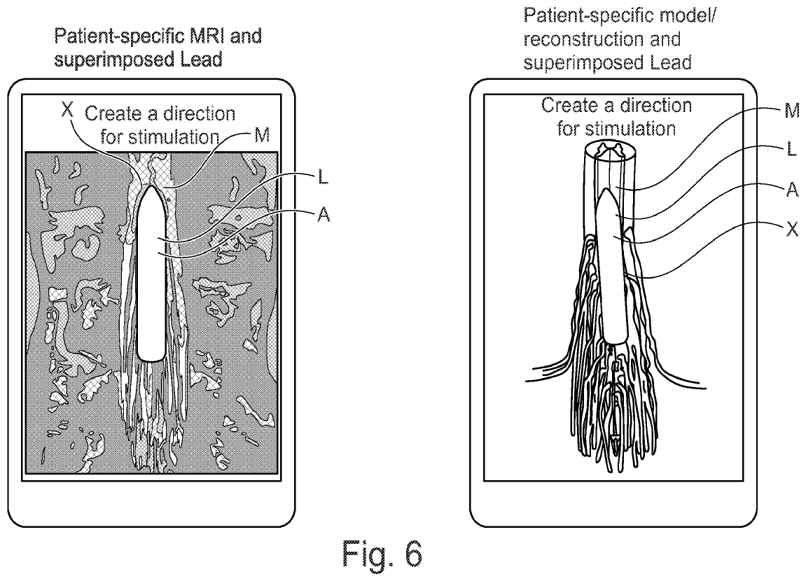

In some embodiments (not depicted in FIG. 1), system 10 may further comprise at least one computer-assisted module configured and arranged for at least partially automatically determining a stimulation zone Z and/or a stimulation direction D on the electrode array A comprising at least one electrode E, V and/or for at least partially automatically selecting at least one electrode E, V, optionally based on medical imaging superimposing (e.g., as described below with regards to FIG. 6).

In some embodiments (not depicted in FIG. 1), calculation module 16 could be configured and arranged to consider power efficiency in determining a weighted contribution of currents.

Based on the electrodes E, V of the stimulation zone Z and/or the stimulation direction D and/or the at least one electrode E, V individually selected and/or the at least one target area X indicated by the user, the calculation module 16 may use a (pre-computed, or online calculated) neuronal activation model, to optimize the weighting of electrode E, V intensities for maximizing the selectivity index, or for maximizing the target while minimizing the sensitive areas.

In some embodiments, system 10 may perform a method for planning neuromodulation, including neurostimulation, at least comprising the steps of: providing graphical information about an electrode array A comprising multiple electrodes E, V and/or an implantation side for the electrode array A comprising at least one target area X, determining a stimulation zone Z and/or a stimulation direction D on the electrode array A comprising at least one electrode E, V and/or individually selecting at least one electrode E, V and/or selecting at least one target area X, determining a contribution of currents provided by electrodes E, V of the stimulation zone Z and/or the stimulation direction D on the electrode array A and/or the at least one electrode E, V selected and/or to the at least one selected target area X.

In general, the graphical information about the electrode array A may comprise physical electrodes E and/or virtual electrodes V.

The method may further comprise the step of determining an equal contribution of currents provided by the electrodes E, V of the stimulation zone Z and/or the stimulation direction D and/or at least one electrode E, V individually selected and/or to the at least one target area X selected.

The method may further comprise the step of determining a weighted contribution of currents provided by the electrodes E, V of the stimulation zone Z and/or the stimulation direction D and/or at least one electrode E, V individually selected and/or to the at least one target area X selected.

The method may further comprise the step of determining the weighted contribution of currents provided by the electrodes E, V of the stimulation zone Z and/or the stimulation direction D by calculating the Euclidean distance from an electrode E, V to the stimulation zone Z and/or the stimulation direction D and/or to at least one point of the stimulation zone Z and/or stimulation direction D.

The method may further comprises the step of determining the weighted contribution of currents provided by the electrodes E, V of the stimulation zone Z and/or the stimulation direction D and/or the at least one electrode E, V individually selected based on a generated field of neighbor electrodes E, V.

The method may further comprise the step of determining the weighted contribution of currents provided by the electrodes E, V of the stimulation zone Z and/or the stimulation direction D and/or at least one electrode E, V individually selected and/or to the at least one target area X selected by a numerical method.

The method may further comprise the step of least partially automatically determining a stimulation zone Z and/or a stimulation direction D on the electrode array A comprising at least one electrode E, V and/or for at least partially automatically selecting at least one electrode E, V, optionally based on medical imaging superimposing.

The method may consider power efficiency in determining the weighted contribution of currents. In general, the weighting may be presented as absolute numbers (e.g. in V, mA, A) and/or expressed as percentage of total current applied by involved electrodes E, V.

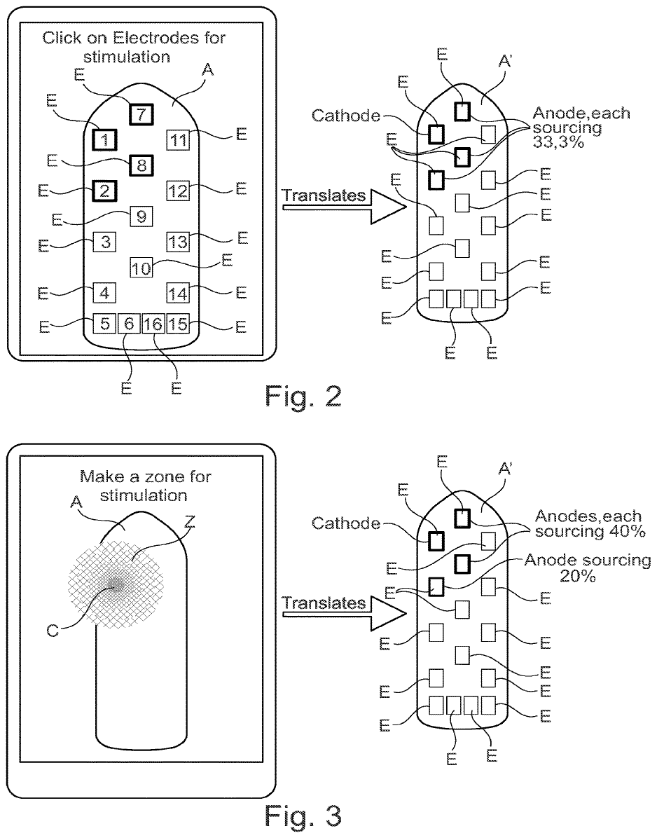

Examples of how to determine a stimulation zone Z and/or stimulation direction D and/or select individual electrodes E, V and/or at least one target area X are disclosed in FIGS. 2, 3, 4, 5, 6, 7 and/or 8.

FIG. 2 shows an example of an equal contribution of currents provided by individually selected electrodes E, V of an electrode array A, determined by the calculation module 16 of the system 10 disclosed in FIG. 1. In this non-limiting example, the graphical information provided by graphical presentation module 12 of system 10 includes a depiction of an electrode array A comprising 16 electrodes E. System 10 can be connected to a neuromodulation system, the system including an electrode array A′ and configured and arranged to provide neuromodulation to a patient. The electrode array A depicted in FIG. 2 can represent the electrode array A′ of this connected neuromodulation system.

As depicted in FIG. 2, a tablet computer display that provides a graphical user interface (e.g., a touch screen) can be a selection module for system 10 (e.g., selection module 14). The disclosed embodiments are not so limited: any display and/or touch screen and/or programmer and/or mobile device can implement a selection module for system 10.

In some embodiments, the display can disclose the graphical information provided by the graphical presentation module 12. In some embodiments, a user can select electrodes E of the electrode array A′ by touching corresponding electrodes E of the electrode array A displayed on the touch screen. Alternatively, the electrodes E could be selected via a mouse click. In general, the electrodes E may be selected by a mouse and/or a trackball and/or a joystick, a display and/or a touch screen and/or a touch pad and/or an acoustic signal and/or acoustic tone and/or a verbal command input. In general, the selection module 14 may allow a user to actuate at least one control element, including but not limited to axes, points, knots, buttons, arrows, hand signals, emojis, crosses and/or windows and/or text and/or shortcuts. In some embodiments, the user input is translated by the calculation module 16 into a certain weighting that may then be sent to the neuromodulation system.

In some embodiments, the neuromodulation provided by the neuromodulation system can be modified based on the user input. To continue the prior example, three electrodes (e.g., electrodes 7, 8, and 2) can be configured by the user using selection module 14 to serve as anodes. For example, they could have been individually selected through user interactions with the display. Calculation module 16 can allocate an equal amount of current to each of these anodes (e. g,. 33.33% of the current sourced by electrode 1, the cathode in this example). In some embodiments, the related neuromodulation system could then provide stimulation to a patient using the electrode array A′ and the determined current allocations.

FIG. 3 shows an example of weighted contribution of currents provided by electrodes E of an electrode array A. In this nonlimiting example, the weighted contribution of currents establishes a stimulation zone Z. The weights of the contribution of currents can be determined by the calculation module 16. The stimulation zone Z can be determined through user interactions with graphical information provided by graphical presentation module 12. In this example, the graphical information can include a depiction of an electrode array A, which includes 16 electrodes E. The electrode array A can represent an electrode array A′ of a neuromodulation system configured and arranged for providing neuromodulation to a patient.

As depicted in FIG. 3, a tablet computer display that provides a graphical user interface (e.g., a touch screen) can be a selection module for system 10 (e.g., selection module 14). In some embodiments, the display can implement a graphical presentation module (e.g., graphical presentation module 12). The disclosed embodiments are not so limited: any display and/or touch screen and/or programmer and/or mobile device can implement a selection module or graphical presentation module for system 10. Selection module 14 can be used to determine at least one stimulation zone Z (e.g., based on a user input).

In some embodiments, a user can select a stimulation zone Z by touching a corresponding location displayed on the touch screen. Alternatively, the stimulation zone could be determined via a mouse click. In general, the stimulation zone and/or a stimulation direction D may be determined through user interactions with a mouse and/or a trackball and/or a joystick, a display and/or a touch screen and/or a touch pad and/or an acoustic signal and/or acoustic tone and/or a verbal command input. In general, a user could interact with selection module 14 to actuate at least one control element, including but not limited to axes, points, knots, buttons, arrows, hand signals, emojis, crosses and/or windows and/or text and/or shortcuts. In some instances, the user input can cause a modification of existing stimulation parameters or existing neuromodulation.

Calculation module 16 can be configured to translate user input obtained using selection module 14 into stimulation parameters. In some instances, when system 10 is connected to a system for neuromodulation, the stimulation parameters may be sent to the neuromodulation system. The stimulation parameters can configure the neuromodulation system to provide neuromodulation according to the user input obtained using selection module 14. In some embodiments, the neuromodulation system can include an electrode array A′. The electrode array A′ of the neuromodulation system could provide stimulation to a patient based on the determined stimulation parameters.

In some embodiments, calculation module 16 can translate the selected stimulation zone Z of an electrode array A into current weightings for physical electrodes E of the electrode array A′. In some embodiments, the weighted contribution of currents provided by the electrodes E may be determined by calculating the Euclidean distance from the electrodes E to the center C of the stimulation zone Z. In some embodiments, the weighted contribution of currents provided by the electrodes E can be determined by calculating the Euclidean distance from the electrodes E to any other point on the electrode array A, including the stimulation zone Z.

In some embodiments, calculation module 16 can determine current weightings for the electrodes of the electrode array A′. In the non-limited example provided in FIG. 3, the calculation module 16 determined a weighted contribution of currents provided by the electrodes E to implement the stimulation zone Z. This weighted contribution includes two anodes (each with 40% of the current) and one anode with 20% of the current (e.g., adding up to 100% of the current provided by the cathode). As would be appreciated by those of skill in the art, this example is not intended to be limiting.

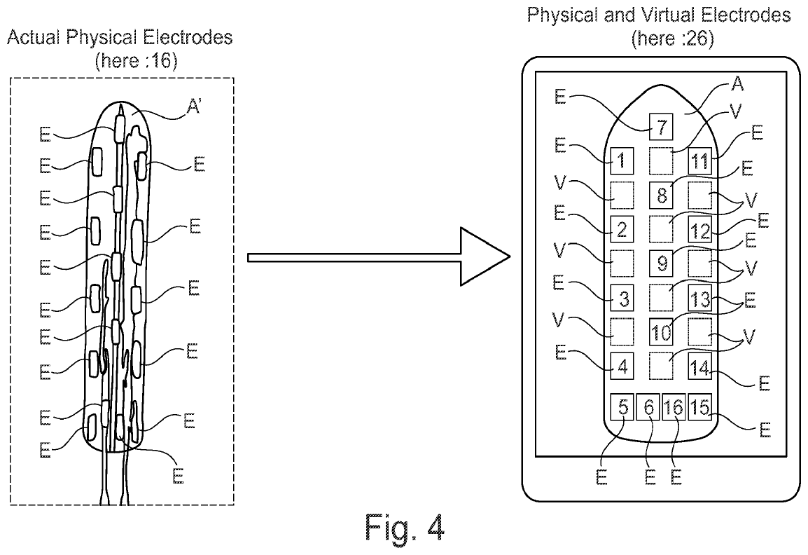

FIG. 4 shows an example of an electrode array A that includes virtual electrodes V. In this nonlimiting example, the electrode array A can represent an electrode array A′ of a neuromodulation system configured and arranged for providing neuromodulation to a patient. The electrode array A′ can include a number of physical electrodes E (e.g., 16 physical electrodes in this example). The electrode array A can include 16 electrodes corresponding to the electrodes of electrode array A′ and 10 virtual electrodes. In some embodiments, the virtual electrodes may not correspond to individual physical electrodes of A′. Instead, each virtual electrode may correspond to a weighted combination of currents provided by at least some of the physical electrodes E. Providing the weighted combination of currents through the at least some of the physical electrodes E may be equivalent to providing current through an electrode situated at the location of the virtual electrode. Electrode array A could alternatively include a different number of virtual electrodes.

As depicted in FIG. 4, a tablet computer can implement a selection module (e.g., selection module 14) and a graphical presentation module (e.g., graphical presentation module 12) for system 10. A display of the tablet computer can implement a graphical presentation module (e.g., graphical presentation module 12). The display can provide graphical information such as a graphical user interface. In this example, the display can depict electrode array A. In some embodiments, the display of the tablet computer can implement a selection module (e.g., selection module 14). As a non-limiting example, the display can be a touchscreen. The selection module can support user interactions that specify or select virtual electrodes V for electrode array A. The disclosed embodiments are not limited to implementation using a tablet computer: any display and/or touch screen and/or programmer and/or mobile device can implement a selection module or graphical presentation module for system 10.

In some embodiments, a user can specify or select virtual electrodes V for electrode array A by touching a corresponding icon or location displayed on the touch screen. Alternatively, the specification or selection of the virtual electrodes V can be determined via a mouse click. In general, the stimulation zone and/or a stimulation direction D may be determined through user interactions with a mouse and/or a trackball and/or a joystick, a display and/or a touch screen and/or a touch pad and/or an acoustic signal and/or acoustic tone and/or a verbal command input. In general, a user could interact with selection module 14 to actuate at least one control element, including but not limited to axes, points, knots, buttons, arrows, hand signals, emojis, crosses and/or windows and/or text and/or shortcuts. In some instances, the user input can cause a modification of existing stimulation parameters or existing neuromodulation.

In some embodiments, the user determines a stimulation zone Z on the electrode array A′ by selecting a combination of physical electrodes E and virtual electrodes V on the electrode array A shown on the display. In some embodiments, the stimulation zone Z can be determined by selecting electrodes E, V via a mouse click. In general, the stimulation zone and/or a stimulation direction D may be determined by a mouse and/or a trackball and/or a joystick, a display and/or a touch screen and/or a touch pad and/or an acoustic signal and/or acoustic tone and/or a verbal command input.

In some embodiments, calculation module 16 can translate user input (e.g., selection of physical electrodes E and virtual electrodes V on an electrode array A, or the like) into simulation parameters (e.g., current weightings, or the like) that can be sent to the neuromodulation system. The neuromodulation system can provide stimulation according to the provided stimulation parameters using the physical electrodes E of electrode array A′. In this manner, a user can configure the stimulation using virtual electrodes, rather than current-weightings or current-steering in percentages. Configuration using virtual electrodes may be easier to comprehend and tune than configuration using current-weightings or current-steering in percentages.

In some embodiments, (not shown in FIG. 4), at least one key performance indicator, such as a selectivity index could be provided as graphical information provided by the graphical presentation module 12. The at least one key performance indicator can be provided before, during, or after selecting and/or interacting with electrodes E and virtual electrodes V of electrode array A. In some embodiments, the at least one key performance indicator can be provided dynamically (e.g., the value of the at least one key performance indicator can be repeatedly or continuously updated during such selection and/or interaction). In some embodiments, the value of the at least one key performance indicator can be determined based on at least one of an anatomical patient model or a neuronal activation model.

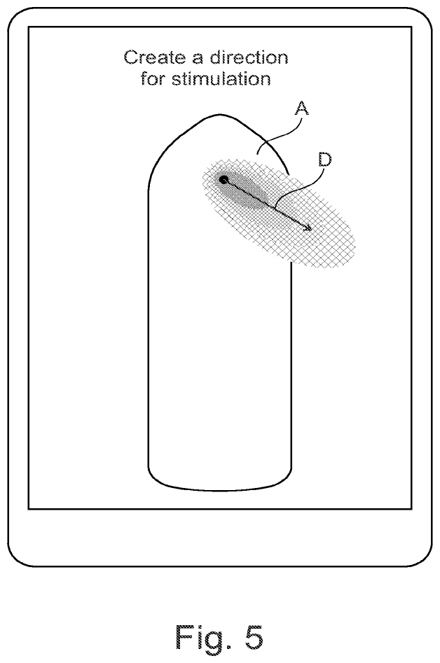

FIG. 5 shows an example of determining a stimulation direction D, according to the disclosed embodiments. In this nonlimiting example, a user can interact with a touchscreen to specify the stimulation direction. For example, the user can touch a point on the touchscreen (e.g., using one or more digits, or the like) and drag to create a stimulation vector (e.g., an origin, direction, and extent of the stimulation). In some embodiments, the user can use multiple touches to add addition stimulation directions or modify or refine an existing stimulation direction (e.g., before, during, or after stimulation). The touchscreen can display an electrode array A that represents an electrode array A′ of neuromodulation system configured and arrange to provide stimulation to a patient. A calculation module (e.g., calculation module 16) could be configured to determine stimulation parameters (e.g., selections of one or more electrodes E of electrode array A′ to provide current, current contributions—weighted or equal—provided by such electrodes, or the like) that would result in a current flow in the specified stimulation direction D (e.g., having the specified origin, direction, or extent). The stimulation parameters can be provided to the neuromodulation system to provide the stimulation.

As depicted in FIG. 5, a tablet computer can implement a selection module (e.g., selection module 14) and a graphical presentation module (e.g., graphical presentation module 12) for system 10. A display of the tablet computer can implement a graphical presentation module (e.g., graphical presentation module 12). The display can provide graphical information such as a graphical user interface. In this example, the display can depict an electrode array A and the stimulation direction D. In some embodiments, the display of the tablet computer can implement a selection module (e.g., selection module 14). As a non-limiting example, the display can be a touchscreen. The selection module can support user interactions that specify or select the stimulation direction D. The disclosed embodiments are not limited to implementation using a tablet computer: any display and/or touch screen and/or programmer and/or mobile device can implement a selection module or graphical presentation module for system 10.

In some embodiments, at least one key performance indicator, such as a selectivity index could be provided as graphical information provided by the graphical presentation module 12. The at least one key performance indicator can be provided before, during, or after selecting and/or interacting with stimulation direction D. In some embodiments, the at least one key performance indicator can be provided dynamically (e.g., the value of the at least one key performance indicator can be repeatedly or continuously updated during such selection and/or interaction). In some embodiments, the value of the at least one key performance indicator can be determined based on at least one of an anatomical patient model or a neuronal activation model.

The disclosed embodiments are not limited to embodiments in which the user specifies a stimulation direction D by touching and dragging a digit, or the like. In some embodiments, the specification of the stimulation direction D can be determined via a mouse click. In general, the stimulation direction D may be determined through user interactions with a mouse and/or a trackball and/or a joystick, a display and/or a touch screen and/or a touch pad and/or an acoustic signal and/or acoustic tone and/or a verbal command input. In general, a user could interact with selection module 14 to actuate at least one control element, including but not limited to axes, points, knots, buttons, arrows, hand signals, emojis, crosses and/or windows and/or text and/or shortcuts. In some instances, the user input can cause a modification of existing stimulation parameters or existing neuromodulation.

FIG. 6 shows two views of graphical information provided by the graphical presentation module (e.g., graphical presentation module 12), in accordance with disclosed embodiments. In this non-limiting example, each of the two views combine an electrode array A with a target area X, according to the disclosed embodiments. In the left view (titled “Patient-specific MRI and superimposed lead”) a visualization of the target area X is displayed. The visualization can be based on patient-specific data obtained by Magnetic resonance imaging (MRI), X-Ray, computed tomography (CT) scan, pictures obtained during surgery, or other medical images. In the right view (titled “Patient-specific model/reconstruction and superimposed lead”) a reconstruction or a model M of the target area X is displayed. In various embodiments, the target area X could be provided as a 2D model M and/or a 3D model M. In both views, as depicted in FIG. 6, the electrode array A can be superimposed on the visualization or reconstruction/model. In some embodiments, the electrode array A can be, or be part of a lead L. In such embodiments, as depicted in FIG. 6, the lead L can be superimposed on a target area X.

In some embodiments, a user could interact with a view to place or adjust a location of the electrode array A on at least one target area X by touching a location of electrode array A in the display with a digit and moving the electrode array A with the digit. In some 3D embodiments, the user could be able to control a position and orientation of the array A in three dimensions (e.g., by rotating the view, the array, or both). In various embodiments, a computer-assisted module may at least partially automatically determine a stimulation zone Z and/or a stimulation direction D on the electrode array A comprising at least one electrode E, V and/or for at least partially automatically selecting at least one electrode E, V optionally based on the medical imaging superimposing and/or a model M.

In some embodiments, at least one key performance indicator, such as a selectivity index could be provided as graphical information provided by the graphical presentation module 12. The at least one key performance indicator can be provided before, during, or after placement of the electrode array A on at least one target area X. In some embodiments, the at least one key performance indicator can be provided dynamically (e.g., the value of the at least one key performance indicator can be repeatedly or continuously updated during such placement). In some embodiments, the value of the at least one key performance indicator can be determined based on at least one of an anatomical patient model or a neuronal activation model.

As depicted in FIG. 6, a tablet computer can implement a selection module (e.g., selection module 14) and a graphical presentation module (e.g., graphical presentation module 12) for system 10. A display of the tablet computer can implement a graphical presentation module (e.g., graphical presentation module 12). The display can provide graphical information such as a graphical user interface. In this example, the display can depict a view of the electrode array A superimposed on the visualization or reconstruction/model. In some embodiments, the display of the tablet computer can implement a selection module (e.g., selection module 14). As a non-limiting example, the display can be a touchscreen. The selection module can support user interactions that specify or select the position and/or orientation of at least one of the array or the reconstruction/model. The disclosed embodiments are not limited to implementation using a tablet computer: any display and/or touch screen and/or programmer and/or mobile device can implement a selection module or graphical presentation module for system 10.

The disclosed embodiments are not limited to embodiments in which the user places or adjusts a location of the electrode array A selecting and moving the electrode array A with a digit, or the like. In some embodiments, the placement of electrode array A can be determined via a mouse click. In general, the placement of electrode array A can be determined through user interactions with a mouse and/or a trackball and/or a joystick, a display and/or a touch screen and/or a touch pad and/or an acoustic signal and/or acoustic tone and/or a verbal command input. In general, a user could interact with selection module 14 to actuate at least one control element, including but not limited to axes, points, knots, buttons, arrows, hand signals, emojis, crosses and/or windows and/or text and/or shortcuts. In some instances, the user input can cause a modification of existing stimulation parameters or existing neuromodulation.

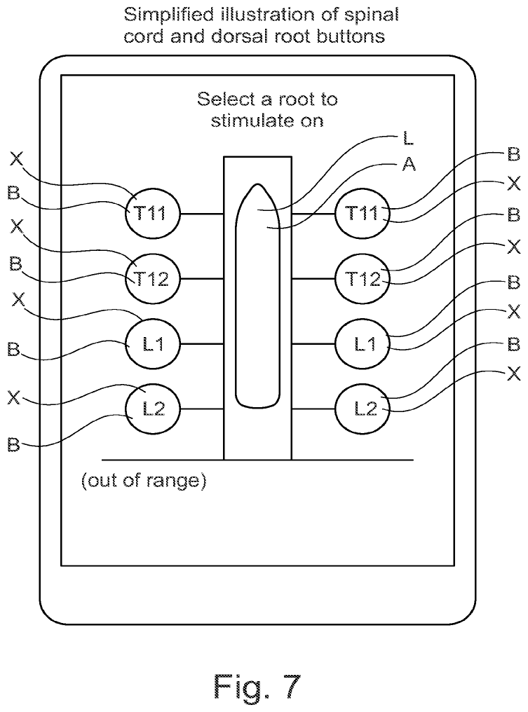

FIG. 7 shows a further example of graphical information provided by the graphical presentation module. In this example, the graphical information can include a schematic depiction. In some embodiments, the schematic depiction could include one or more elements, such as target area(s) X, the electrode array A, or the lead L (e.g., a lead L being or containing electrode array A). In some embodiments, the lead L or array A could be superimposed on one or more other elements. In general, schematic representations of muscles and/or organs could be positioned under an electrode array A and/or a lead L and/or or next to an electrode array A and/or a lead L.

Consistent with disclosed embodiments, icons or controls can represent the different elements depicted in the schematic representation. The icons or controls can be specific to the thing represented. The disclosed embodiments are not limited to any particular icons or representations. In some embodiments, the user can interact with the schematic representation to select an element for stimulation. Selecting the icon or control can cause calculation module 16 to translate the selection of the element into stimulation parameters (e.g., selections of one or more electrodes E of electrode array A′ to provide current, current contributions—weighted or equal—provided by such electrodes, or the like) that would result in stimulation or activation of the selected element. The stimulation parameters can be provided to the neuromodulation system to provide the stimulation.

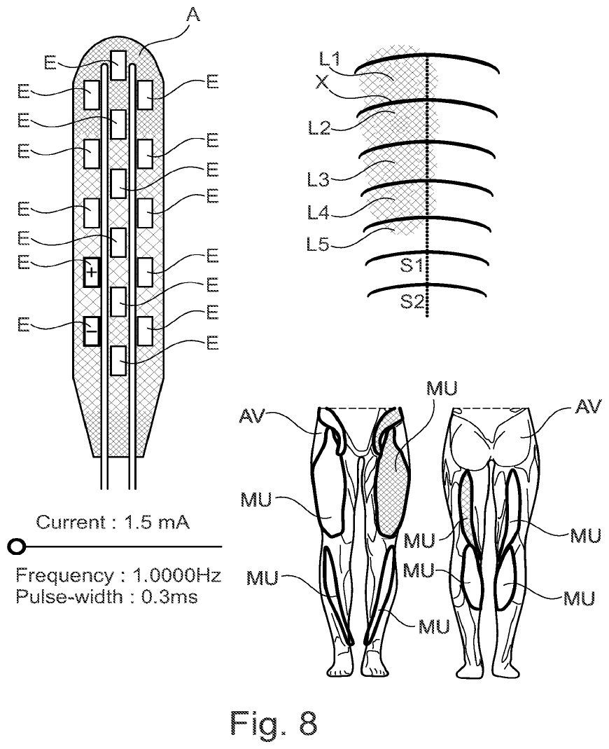

In various embodiments, the elements could include anatomical structures such as nerves, tissues, bones, muscles (e.g. leg/trunk muscles), glands, organs, or other anatomical structures. In some embodiments, the at least one target area X could be or include at least one of target nerve(s); nerve fiber(s); dorsal root(s); spinal cord area(s); tissue(s); organ(s); gland(s); or area(s) related to at least one of the spinal cord, muscle fiber, or muscles. In some embodiments, the schematic depiction could include one or more actions (e.g., movements; bladder, bowel, or sexual functions; or the like). In the same manner as selecting target area(s) X, action(s) could be selected. As described in greater detail below with regards to FIG. 8, stimulation can be applied according to the selected actions.

When selecting actions, the target area X could be alternatively and/or additionally be associated with kinematic model M, wherein intended muscles could be determined and weighted and/or unwanted crosstalk between agonists and antagonists could be assigned. In this manner, actions could be broken down into time-dependent, muscle-specific activation patterns. In this non-limiting example, such activation patterns could be translated into stimulation provided by electrodes E of the electrode array A′.

In some embodiments, the schematic depiction can be or include fictious and/or realistic anatomical conditions of a mammal, in some embodiments a human being, such as the patient himself. In some embodiments, image data and/or 2D models M and/or 3D models M could be used as target area X for visual representation of anatomy of the patient, e.g. the dorsal roots.

As depicted in FIG. 7, for example, the schematic depicts the spinal cord as a rectangle and spinal nerves (e.g., pairs of thoracic nerves T11, T12 and lumbar nerves L1, L2) as buttons. The possible target areas X include the spinal cord and the spinal nerves. In this non-limiting example, the user can select at least one target area X, i.e. left and/or right thoracic nerve T11 and/or T12 and/or left and/or right lumbar nerve L1 and/or L2 by selecting the corresponding buttons B (e.g. by touching the corresponding button B with a digit).

In some embodiments, at least one key performance indicator, such as a selectivity index could be provided as graphical information provided by the graphical presentation module 12. The at least one key performance indicator can be provided before, during, or after selection and/or interaction with target area(s) X. In some embodiments, the at least one key performance indicator can be provided dynamically (e.g., the value of the at least one key performance indicator can be repeatedly or continuously updated during such selection and/or interaction). In some embodiments, the value of the at least one key performance indicator can be determined based on at least one of an anatomical patient model or a neuronal activation model.

As depicted in FIG. 7, a tablet computer can implement a selection module (e.g., selection module 14) and a graphical presentation module (e.g., graphical presentation module 12) for system 10. A display of the tablet computer can implement a graphical presentation module (e.g., graphical presentation module 12). The display can provide graphical information such as a graphical user interface. In this example, the display can depict a view of the schematic representation of the elements. In some embodiments, the display of the tablet computer can implement a selection module (e.g., selection module 14). As a non-limiting example, the display can be a touchscreen. The selection module can support user interactions that select or interact with the elements. The disclosed embodiments are not limited to implementation using a tablet computer: any display and/or touch screen and/or programmer and/or mobile device can implement a selection module or graphical presentation module for system 10.

The disclosed embodiments are not limited to embodiments in which the user selects or interacts with the schematic depiction using a touchscreen. In some embodiments, the selection or interaction can be determined via a mouse click. In general, the selection or interaction can be determined through user interactions with a mouse and/or a trackball and/or a joystick, a display and/or a touch screen and/or a touch pad and/or an acoustic signal and/or acoustic tone and/or a verbal command input. In general, a user could interact with selection module 14 to actuate at least one control element, including but not limited to axes, points, knots, buttons, arrows, hand signals, emojis, crosses and/or windows and/or text and/or shortcuts. In some instances, the user input can cause a modification of existing stimulation parameters or existing neuromodulation.

FIG. 8 shows a further example of graphical information provided by the graphical presentation module 12, according to the disclosed embodiments. In some embodiments, the graphical information provided by the graphical presentation module 12 includes possible target muscles MU to be stimulated, illustrated as different muscles MU of an avatar AV. In some embodiments, the graphical information provided by the graphical presentation module 12 further comprises a schema configured and arranged to illustrate a target area X based on target muscles MU. A user can select at least one target muscle MU by selecting at least one muscle MU, e.g. by touching the corresponding muscle MU of the avatar AV with a finger. As a non-limiting example, the user may select the right quadriceps Q. The selection module 14, based on the selected target muscle MU, can select a target area X. The target area X comprises spinal nerves L1-L5. In this non-limiting example, a patient is implanted with an electrode array A (electrode array A also shown in FIG. 8) capable of providing stimulation on the target area X. Calculation module 16 can determine a contribution of currents provided to the target area X selected in order to stimulate the muscle MU selected. In some embodiments, the contribution of currents provided to the target area X can gradually decreases from the center to the outside O of the target area X. The electrode array A provides stimulation to the target area X via selected electrodes E and selected stimulation parameters. In this non-limiting example, the current is 1.5 mA, the frequency is 1.0000 Hz and the pulse-width is 0.3 ms. However, other stimulation parameters could be generally possible.

As depicted in FIG. 8, a tablet computer can implement a selection module (e.g., selection module 14) and a graphical presentation module (e.g., graphical presentation module 12) for system 10. A display of the tablet computer can implement a graphical presentation module (e.g., graphical presentation module 12). The display can provide graphical information such as a graphical user interface. In this example, the display can depict muscles MU of an avatar AV. In some embodiments, the display of the tablet computer can implement a selection module (e.g., selection module 14). As a non-limiting example, the display can be a touchscreen. The selection module can support user interactions that select or interact with the elements. The disclosed embodiments are not limited to implementation using a tablet computer: any display and/or touch screen and/or programmer and/or mobile device can implement a selection module or graphical presentation module for system 10.

The methods of stimulation planning disclosed in FIGS. 2 to 8 can be combined or performed using a single system. For example, a user could select a target muscle (as in FIG. 8) or a target area (as in FIG. 7), or position the electrode (as in FIG. 6). The user could then cause the system to display the stimulation associated with that selection in terms of electrodes and weights (as in FIG. 2), stimulation zones (as in FIG. 3), virtual electrodes (as in FIG. 4), or stimulation directions (as in FIG. 5). The stimulation could then be refined or updated as described with regards to each of these figures. Such updating or refinement can be performed before, during, or after provision of the stimulation.

It will be appreciated that the configurations and routines disclosed herein are exemplary in nature, and that these specific embodiments are not to be considered in a limiting sense, because numerous variations are possible. The subject matter of the present disclosure includes all novel and non-obvious combinations and sub-combinations of the various systems and configurations, and other features, functions, and/or properties disclosed herein.

The following claims particularly point out certain combinations and sub-combinations regarded as novel and non-obvious. Such claims should be understood to include incorporation of one or more such elements, neither requiring nor excluding two or more such elements. Other combinations and sub-combinations of the disclosed features, functions, elements, and/or properties may be claimed through amendment of the present claims or through presentation of new claims in this or a related application. Such claims, whether broader, narrower, equal, or different in scope to the original claims, are also regarded as included within the subject matter of the present disclosure.

What is claimed is:

1. A planning and/or control system for a system for providing neuromodulation, comprising:
a graphical presentation module configured and arranged for providing graphical information about an electrode array comprising multiple electrodes and/or an implantation side for the electrode array comprising at least one target area,
a selection module configured and arranged for determining a stimulation zone and/or a stimulation direction on the electrode array comprising at least one electrode and/or for individually selecting at least one electrode and/or for selecting at least one target area,
a calculation module configured and arranged for determining a contribution of currents provided by electrodes of the stimulation zone and/or stimulation direction on the electrode array and/or the at least one electrode selected and/or to the at least one target area selected.

2. The system according to claim 1, wherein the graphical presentation module is configured and arranged for providing graphical information about the electrode array comprising actual physical electrodes and/or virtual electrodes.

3. The system according to claim 2, wherein the calculation module is configured and arranged for determining an equal contribution of currents provided by the electrodes of the stimulation zone (Z) and/or stimulation direction and/or the one or more electrodes individually selected and/or to the at least one target area selected.

4. The system according to claim 2, wherein the calculation module is configured and arranged for determining a weighted contribution of currents provided by the electrodes of the stimulation zone and/or stimulation direction and/or the one or more electrodes individually selected and/or to the at least one target area selected.

5. The system according to claim 4, wherein the calculation module is configured and arranged for determining the weighted contribution of currents provided by the electrodes of the stimulation zone and/or stimulation direction by calculating a Euclidean distance from an electrode to the stimulation zone and/or stimulation direction and/or to at least one point of the stimulation zone and/or stimulation direction.

6. The system according to claim 4, wherein the calculation module is configured and arranged for determining the weighted contribution of currents provided by the electrodes of the stimulation zone and/or the stimulation direction and/or at least one electrode individually selected based on a generated field of neighbor electrodes.

7. The system according to claim 4, wherein the calculation module is configured and arranged for determining the weighted contribution of currents provided by the electrodes of the stimulation zone and/or the stimulation direction and/or the at least one electrode individually selected and/or to the at least one target area selected by a numerical method.

8. The system according to claim 1, wherein the system further comprises at least one of a display, a controller, a programmer, a communication module, a telemetry module, a stimulation device, an electrode, a sensor and/or a sensor network.

9. The system according to claim 1, wherein the system further comprises at least one computer-assisted module configured and arranged for at least partially automatically determining a stimulation zone and/or a stimulation direction on the electrode array comprising at least one electrode and/or for at least partially automatically selecting at least one electrode.

10. The system according to claim 7, wherein the calculation module is be configured and arranged to feature an algorithm to determine the weighted contribution of currents to a benefit of power efficiency.

11. A method for planning neuromodulation comprising the steps of:
providing graphical information about an electrode array comprising multiple electrodes and/or an implantation side for the electrode array comprising at least one target area,
determining a stimulation zone and/or a stimulation direction on the electrode array comprising at least one electrode and/or individually selecting at least one electrode and/or selecting at least one target area,
determining a contribution of currents provided by electrodes of the stimulation zone and/or the stimulation direction on the electrode array and/or the at least one electrode selected and/or to at least one selected target area.

12. The method according to claim 11, wherein the graphical information about the electrode array comprises actual physical electrodes and/or virtual electrodes.

13. The method according to claim 12, wherein the method further comprises a step of determining an equal contribution of currents provided by the electrodes of the stimulation zone and/or the stimulation direction and/or at least one electrode individually selected and/or to the at least one target area selected.

14. The method according to claim 12, wherein the method further comprises a step of determining a weighted contribution of currents provided by the electrodes of the stimulation zone and/or the stimulation direction and/or at least one electrode individually selected and/or to the at least one target area selected.

15. The method according to claim 14, wherein the method further comprises the step of determining the weighted contribution of currents provided by the electrodes of the stimulation zone and/or the stimulation direction by calculating a Euclidean distance from an electrode to the stimulation zone and/or the stimulation direction and/or to at least one point of the stimulation zone and/or at least one stimulation direction.

16. The method according to claim 14, wherein the method further comprises the step of determining the weighted contribution of currents provided by the electrodes of the stimulation zone and/or the stimulation direction and/or the at least one electrode individually selected based on a generated field of neighbor electrodes.

17. The method according to claim 14, wherein the method further comprises the step of determining the weighted contribution of currents provided by the electrodes of the stimulation zone and/or the stimulation direction and/or at least one electrode individually selected and/or to the at least one target area selected by a numerical method.

18. The method according to claim 10, wherein the method further comprises the step of least partially automatically determining a stimulation zone and/or a stimulation direction on the electrode array comprising at least one electrode and/or for at least partially automatically selecting at least one electrode.

19. The method according to claim 18, wherein the method further comprises the step of featuring an algorithm to determine the weighted contribution of currents to the benefit of power efficiency.
